(19) United States
(12) Patent Application Publication
(10) Pub. No.: US 2011/0184472 A1
(43) Pub. Date: Jul. 28, 2011

(54) Expandable Bone Support

(76) Inventors: Alfred Niederberger, Salzburg (AT); Johann Fierlbeck, Salzburg (AT)

(21) Appl. No.: 13/122,037

(22) PCT Filed: Sep. 14, 2009
(86) PCT No.: PCT/US09/56797

§ 371 Date: Mar. 31, 2011

Related U.S. Application Data
(60) Provisional application No. 61/103,375, filed on Oct. 7, 2008.

(51) Int. Cl. A61B 17/86

(52) U.S. Cl. 606/304; 606/319

(57) ABSTRACT

A bone fixation device comprises a rod extending from a proximal end located external to the body in an operative configuration to a distal end comprising an increased diameter tip sized and shaped to be received in a subchondral layer in the operative configuration and a first bushing slidably received over the rod, a distal portion of the first bushing comprising a plurality of arms separable from one another to spread radially outward from the rod as the first bushing is moved distally over the tip of the rod in combination with a cannulated element slidably received over the rod, the cannulated element sized and shaped to engage a proximal end of the first bushing, a portion thereof located external to the bone in the operative configuration so that a distally directed force applied thereto moves the first bushing distally over the rod spreading the arms radially outward.

### PRIORITY CLAIM

This application claims the benefit of U.S. Application Ser. No. 61/103,375 entitled “Expandable Bone Support” filed Oct. 7, 2008, the disclosure of which is incorporated herein by reference in its entirety.

### FIELD OF THE INVENTION

The present invention relates generally to a device for the fixation of fractured or otherwise damaged bone and, in particular, relates to a device which expand after insertion into the bone to enhance the structural integrity of the bone.

### BACKGROUND

Various implants are used to stabilize portions of bone after a fracture. These implants include, for example, longitudinal load carriers (e.g., plates or rods) that are usually manufactured from one material such as stainless steel, titanium or its alloys. Longitudinal load carriers are generally secured to the portions of the fractured bone via bone fixation elements such as screws, pins or rivets. However, bone degradation, such as that caused by osteoporosis, may require that the fixation elements counterbalance weakened bone structures unable to otherwise support the longitudinal load carrier.

### SUMMARY OF THE INVENTION

The present invention is directed to a bone fixation device comprising a rod extending from a proximal end located external to the body in an operative configuration to a distal end comprising an increased diameter tip sized and shaped to be received in a subchondral layer in the operative configuration and a first bushing slidably received over the rod, a distal portion of the first bushing comprising a plurality of longitudinally extending arms, the arms being separable from one another to spread radially outward from the rod as the first bushing is moved distally over the tip of the rod in combination with a cannulated element slidably received over the rod, the cannulated element sized and shaped to engage a proximal end of the first bushing with a portion thereof located external to the bone and accessible to a user in the operative configuration, so that a distally directed force applied thereto moves the first bushing distally over the rod spreading the arms radially outward relative to the rod.

### BRIEF DESCRIPTION OF THE DRAWINGS

FIG. 1 shows a partial cross-sectional view of a bone fixation device according to the present invention in an insertion configuration in a bone;

FIG. 2 shows a partial cross-sectional view of the bone fixation device of FIG. 1 in an extended configuration in a bone;

FIG. 3 shows a view of a rod of the bone fixation device of FIG. 1;

FIG. 4 shows a bushing of the bone fixation device of FIG. 1;

FIG. 5 shows a tube for expansion of the bone fixation device of FIG. 1;

FIG. 6 shows a cannulated screw for expansion of the bone fixation device of FIG. 1;

FIG. 7A shows a first side view of the bone fixation device of FIG. 1;

FIG. 7B shows a second side view of the bone fixation device of FIG. 1;

FIG. 8A shows a first side view of the bone fixation device of FIG. 1 prior to assuming an expanded configuration;

FIG. 8B shows a second side view of the bone fixation device of FIG. 8A;

FIG. 9A shows a first side view of the bone fixation device of FIG. 1 in an expanded configuration;

FIG. 9B shows a second side view of the bone fixation device of FIG. 9A;

FIG. 10A shows a third side view of the bone fixation device of FIG. 1 in an expanded configuration;

FIG. 10B shows a fourth side view of the bone fixation device of FIG. 10A;

FIG. 11A shows a first side view of the bone fixation device of FIG. 1 prior to assuming an expanded configuration;

FIG. 11B shows a second side view of the bone fixation device of FIG. 11A;

FIG. 12A shows a first side view of the bone fixation device of FIG. 1 in an expanded configuration;

FIG. 12B shows a second side view of the bone fixation device of FIG. 12A;

FIG. 13A shows a partial cross sectional view of the bushing according to a second exemplary embodiment of the present invention in a closed configuration;

FIG. 13B shows a partial cross sectional view of the bushing of FIG. 13A in an expanded configuration;

FIG. 14A shows a frontal view of the bushing of FIG. 13A;

FIG. 14B shows a frontal view of the bushing of FIG. 13B; and

FIG. 15 depicts an alternate embodiment of a cannulated screw according to the present invention.

### DETAILED DESCRIPTION

The present invention may be further understood with reference to the following description and the appended drawings, wherein like elements are referred to with the same reference numerals. The present invention relates generally to methods and devices for the stabilization and fixation of fractured bones and bone fragments. Specifically, the present invention relates to methods and devices for the flexible fixation of extra-articular or articular fractures in portions of the proximal humerus, proximal femur, distal femur, proximal tibia and distal tibia weakened, for example, through osteoporosis. Embodiments of the present invention may also be employed with a plurality of other bone fractures where close contact to the cortical bone is relevant and poor mechanical bone quality is evident such as, for example, in spine surgery and orthopedic surgery. Embodiments of the present invention may also be employed with locking compression plates such as those described in U.S. Pat. No. 6,206,881 to Frigg et al. entitled “Bone Plate”, the entire disclosure of which is expressly incorporated herein by reference.

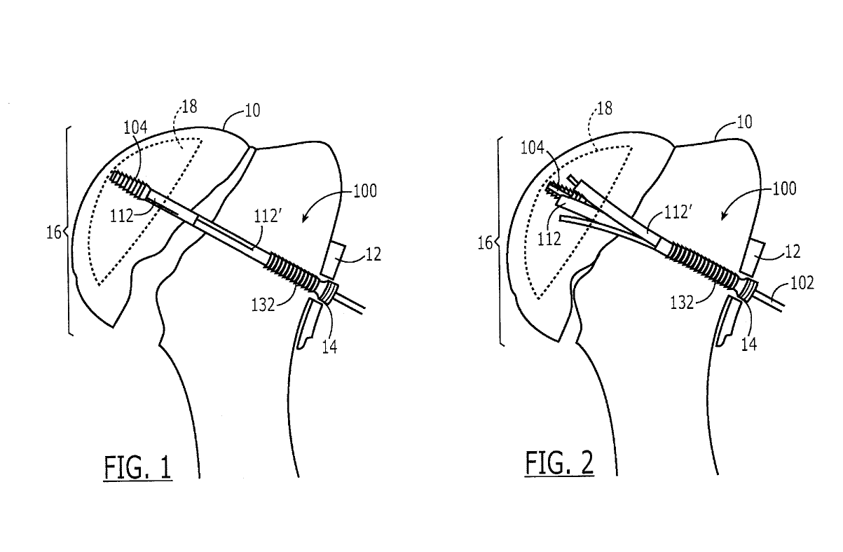

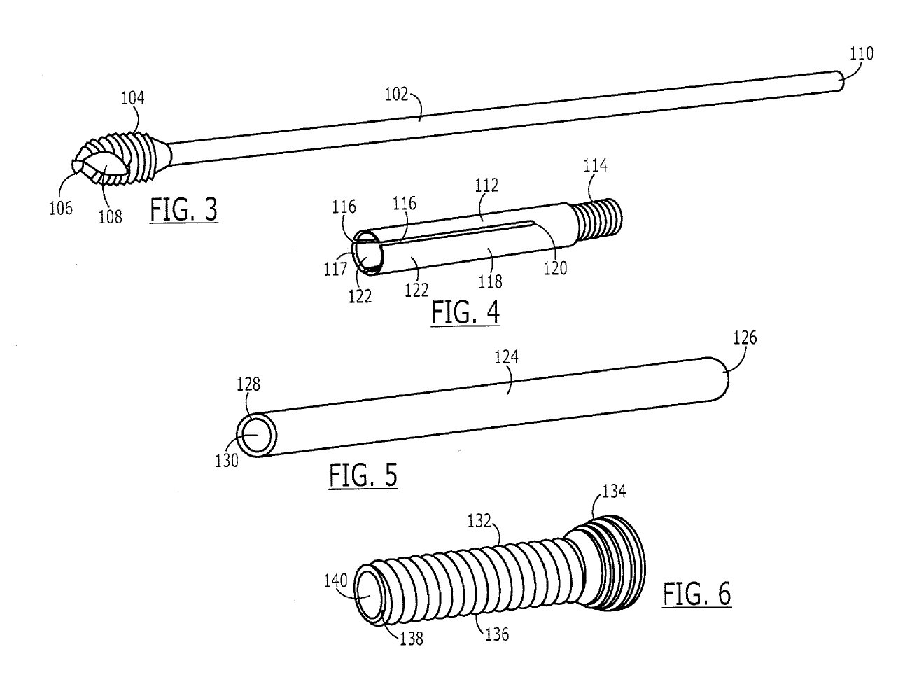

As shown in FIGS. 1-6, a bone fixation device 100 according to one embodiment of the present invention is inserted into a fractured bone 10 past a subchondral layer 18 which, for example, has not been weakened. A portion of the subchondral layer 18 is shown in phantom in embodiments of FIGS. 1 and 2. Those skilled in the art will understand that the subchondral layer 18 extends over substantially the entirety of an epiphysis 16 of the bone 10 and that a reduction in bone mineral density or other weakening may occur to this portion of the bone 10 (e.g., through osteoporosis). In order to properly fix a fracture of such weakened bone, a bone fixation device 100 according to the present invention anchors into the subchondral layer 18 of the bone 10.

The bone fixation device 100 is further adapted to minimize stress concentrations in fractured portions of the bone to which they are attached. Presently available bone fixation devices may exert loads on fractured bone which increase during motion. The increased load may cause weakened bone to collapse, potentially pushing the rigid bone fixation device through the already weakened bone structure before healing has completed. The bone fixation device 100 of the present invention reduces the stiffness of a load-carrying portion thereof to provide a flexible system less likely to result in such inadvertent cutting of the bone. Furthermore, the bone fixation device 100 of the present invention minimizes concentrations of the stress placed on the bone at the implant-bone interface to effectively counterbalance the effects of bone degeneration.

As shown in FIG. 1, the bone fixation device 100 is inserted into the bone 10 so that a distal end thereof is received within the subchondral layer 18. As will be described in greater detail below, the bone fixation device 100 comprises an elongated rod 102 with optional first and second bushings 112, 112′ received over a distal portion thereof. A proximal end of the body 118, 118′ of the respective first and second bushings 112, 112′ may further comprise a slight taper to aid in a sliding of the proximally located bushing thereover. For example, a slight taper (not shown) provided on a proximal end of the body 118 may aid in sliding the second bushing 112′ distally thereover to expand in the subchondral bone, as will be described in greater detail below.

The rod 102 may be formed, for example, as an elongated wire with the threaded distal tip 104 having a diameter as great as and preferably greater than a diameter of the rod 102. A greater diameter of the tip 104 compared to the rod 102 indicates to the user which area of the bone the tip 104 is in. Because subchondral bone is denser than the osteoporosis affected bone, the torque will be higher to screw in the tip 104. In an alternate embodiment (not shown), the tip 104 may not include the threading and may be formed as a self-drilling tip, as those skilled in the art will understand. A distal-most portion of the threaded tip 104 forms a point 106 which aids in screwing the rod 102 into a target portion of the bone 10. The tip 104 comprises threads substantially perpendicular to a longitudinal axis defined by the rod 102 and formed in a helical configuration. The tip 104 further comprises a notch 108 extending substantially parallel to a longitudinal axis of the rod 102. The notch 108 allows for bone tissue regrowth therein so that, once a fracture has been healed, rotation of the rod 102 within the bone is prevented. Furthermore, it is noted that although the rod 102 is shown as a unitary element, longitudinal lengths of the rod 102 and the threaded tip 104 may be formed of one or more parts of the same or different materials as would be understood by those skilled in the art. Components of the bone fixation device 100 of the present invention may be formed of one or more of Cobalt-Chromium-Molybdenum (“CCM”) alloys, titanium alloys, stainless steel alloys, zirconium oxide ceramics, polymers of the Peak family (e.g., PEEK, PEKK, PEK, PEK-EKK, etc.) and bio-absorbable materials (e.g., magnesium alloys, polylactide formulations, etc.).

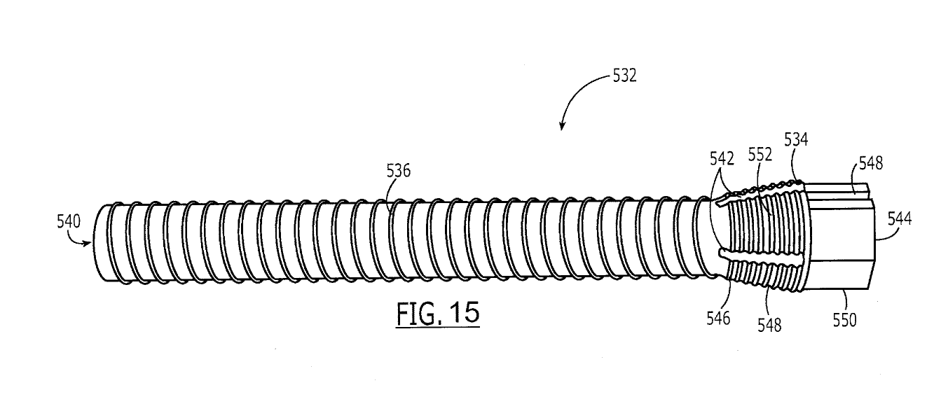

The first bushing 112 is formed as an elongated hollow tube adapted to be received over the rod 102. A proximal end of the first bushing 112 comprises a threaded portion 114 with an outer diameter slightly smaller than an outer diameter of a proximal portion of the body 118 of the first bushing 112 so that, when screwed into a cannulated screw 132 as described below, an outer surface of the proximal portion of the body 118, the outer surfaces of the body 118 and the screw 132 are substantially continuous. In one embodiment, the outer diameter of the proximal portion of the body 118 is approximately 3-5 mm so that the outer diameter of the threaded portion 114 would be smaller by an amount substantially corresponding to a thickness of the screw 132. A distal end 117 of the first bushing 112 comprises a series of slots 116 extending longitudinally into the body 118. The slots 116 extend proximally from the distal end 117 by a predetermined distance to termination points 120. In a preferred embodiment, the longitudinal length of the slots 116 is approximately 9-13 mm and may more preferably be approximately 11 mm. As would be understood by those skilled in the art, the slots of the second bushing 112′ must be slightly longer than the slots 116 to enable the arms 122′ to reach the subchondral bone. Furthermore, those skilled in the art will understand that if no slots are formed in one or both of the first and second bushings 112, 112′, respectively, such a bushing will operate as a spacer element. In the first configuration, such a spacer element is located between the tip 104 and the tube 124. In a second configuration, the spacer is located between tip 104 and the cannulated screw 132. In these two configurations the bushing has been entirely replaced by the spacer which functions as a damping element preventing the tip 104 from gliding proximally along the rod 102 which is not secured without a clamping device or the screw shown in FIG. 15. Having a plastic material or a bioresorbable material for the spacer will give the whole construct more flexibility. Such a bioresorbable spacer may provide enhanced stability required during an initial healing period. After several weeks when the fracture gap has widened, the bioresorbable material begins dissolving and no longer bears the load. The rod 102 may then glide proximally to close the gap again.

The slots 116 define arms 122 which, as described below, are bent radially outward when the bushing 112 is deployed. Furthermore, it is submitted that, although the exemplary embodiment shown comprises three arms 122, any number of arms may be employed without deviating from the spirit and scope of the present invention. Furthermore, as would be understood by those skilled in the art, the device does not need to include a bushing to operate as desired. These slots must be as long as the tip and the first bushing together to reach the subchondral bone and may, for example, be in the range of 20 mm to 30 mm in length.

The first and second bushings 112, 112′ are adapted to be received over a distal end of the rod 102 with a substantial friction fit to provide rotational stability thereto, wherein the first bushing 112 is located distally of the second bushing 112′. In this manner, the first and second bushings 112, 112′ are only movable along a longitudinal axis of the rod 102 when a predetermined force is applied thereto. In an exemplary embodiment of the present invention, one or more bushings may be provided over the rod 102 in order to increase the structural integrity of the bone fixation device 100 in the fractured bone 10.

The rod 102 and the bushings 112, 112′ may be formed, for example, of any suitable biocompatible metal known in the art. For example, the bushing may be manufactured out of a biocompatible polymer which facilitates removal of the entire construct during the procedure, if necessary. The bushing would be destroyed by pulling or screwing back the rod 102 which would not be possible with a metal bushing. The material for the rod 102 and the first bushing 112 may be selected based on a rigidity required to perform a particular procedure. The material of the rod 102 will also serve as an indicator for the stiffness and twist of the bone fixation device 100 when employed in the body. Specifically, because the bone fixation element 100 is composed of a plurality of elements coupled to one another, the bone fixation device 100 is more flexible than a similar device formed as a unitary unit. Furthermore, the rod 102, which is adapted to be screwed into the target portion of the bone, can comprise a material that will affect the twist thereof during the screwing process.

When a cannulated screw 132 is screwed over the rod 102, a pressure is applied to both the first and second bushings 112, 112′, causing the first and second bushings 112, 112′ to slide distally along the rod 102 toward a threaded, increased diameter distal tip 104 thereof. As will be described in more detail below, as the rod 102 is screwed into the bone, the user will be able to feel a difference in the twisting or winding-up of the rod 102 depending on the softness of the bone into which the tip 104 is penetrating. That is, as the tip 104 is larger than the portion of the rod extending proximally therefrom, the amount of wind-up of the rod 102 will increase significantly as the tip 104 encounters harder bone. This will provide tactile feedback enabling the user to determine when the tip 104 has entered the subchondral layer 18. After the tip 104 has been inserted to a desired position in the subchondral layer 18, the first and second bushings 112, 112′, each of which is separated into a plurality of arms 122, 122′, respectively, are moved distally over the rod 102 so that the enlarged tip 104 spreads the arms 122, 122′ radially outward to engage a wider area of the bone, as shown in FIG. 2.

In use, only a distal portion of the rod 102 is adapted to be received in the bone 10. Specifically, as shown in FIGS. 1 and 2, the rod 102 is traversed into the bone 10 so that the threaded tip 104 is located in the subchondral layer 18. The rod 102 may be screwed into the bone 10 in any known manner. For example, the rod 102 may be manually screwed into the bone via manipulation of a handle (not shown) located at a proximal end of the rod 102. As would be understood by those skilled in the art, a bore will first be formed through the cortex of the bone allowing the rod 102 to penetrate into the bone. Further drilling into the spongious bone may not be required. However, whether the rod 102 is moving through a previously formed bore or penetrating the bone for the first time, the tactile feedback will still enable the user to determine when the distal tip 104 has entered a harder layer of bone as described below. That is, as the rod 102 approaches the substantially harder cortical bone near a joint of the bone 10, the rod 102 twists in response to pressure exerted thereto by the cortical bone. The twisting of the rod 102 provides clear mechanical feedback to a user of the bone fixation device 100 that the distal threaded tip 104 has reached a target portion of the bone 10 proximate to the subchondral layer 18. Furthermore, the ratio of the diameter of the rod 102 to the diameter of the threaded tip 104 is indicative of the sensibility of feedback to the user, aiding in insertion of the rod 102 to a required depth in the bone 10. Different materials may be selected for the rod 102 to affect the sensibility thereof. The rod 102 of the present invention is thus guided to a designated location of the bone without length determination and the need for x-rays, both presently employed in the art.

The bone fixation device 100 further comprises two elements used to deploy the first and second bushings 112, 112′ from a closed configuration to an extended configuration in the bone 10. According to a first exemplary embodiment, a cannulated screw 132 is employed. The cannulated screw 132 is formed as a hollow screw extending from an increased diameter head 134 formed at a proximal end to a threaded shaft 134 leading to a distal end 138. A cannula 140 extends longitudinally through the cannulated screw 132 to receive the rod 102 therethrough, a diameter of the cannula 140 substantially equal to a diameter of the lumen 130. The head 134 of the cannulated screw 132 comprises a diameter at least greater than a bore formed in the bone 10 by the rod 102 to prevent the screw 132 from entering into the bone beyond a visible distance. As would be understood by those skilled in the art, a distal portion of the inner diameter of the cannulated screw 132 may be provided with optional threading adapted to engage the threaded portion 114 of the bushing 112. For a bushing formed of metal, the threading would provide a mechanism for removing the bushing and the entire device should that become necessary. Such threads would also facilitate the connection of the tube 124 to the cannulated screw 132. Screwing of the cannulated screw 132 applies a pressure to the second bushing 112′, which in turn applies the pressure to the first bushing 112. The distal pressure on the first bushing 112 causes the first bushing 112 to slide over the threaded tip 104 of the rod 102, thus causing arms 122 thereof to splay radially outward as the first bushing 112 is pushed further distally into the bone 10. The distal force applied to the first bushing 112 and thus, fragments of the bone 10, is countered by a counter force applied by the splayed arms 122. It is noted that any sintering caused by the screwing of the cannulated screw 132 into the bone is minimal and does damage the bone or joint surface. Once the first bushing 112 has been moved to an expanded configuration, the pressure applied by the cannulated screw 132 causes the second bushing 112′ to move distally over the first bushing 112 over the tapered proximal end (not shown) of the bushing 112 which facilitates distal movement of the second bushing 112′ by minimizing obstruction thereto from a proximal face of the first bushing 112.

According to a second exemplary embodiment, a tube 124 is employed in place of the cannulated screw 132. The tube 124 comprises an elongated body extending from a proximal end 126 to a distal end 128. A lumen 130 extends longitudinally through the tube 124 and is sized and shaped to receive the rod 102 therethrough with a substantial friction fit similar to the fit of the first bushing 112. The tube 124 may be fitted over the rod 102 proximal to the first bushing 112 and the second bushing 112′ received over a distal portion thereof prior to insertion into the bone. The proximal end 126 of the tube 124 may be tightly connected to the proximal end 110 of the rod 102 via for example, welding or the use of an adhesive. Thus, the rod 102 and tube 124 may be simultaneously inserted to a target position in the bone 10 without a loosening and/or disengagement of the tube 124. Specifically, a distally directed force applied to the first bushing 112 and the second bushing 112′ using any known method acts similarly to the embodiment discussed above where distal movement of the arms 122 over the threaded tip 104 causes the arms 122 to splay radially outward until the termination point 120 comes into contact with the threaded tip 104. Distal movement of the first bushing 112 and the second bushing 112′ in this embodiment is not resisted by any significant counter forces. Once the rod 102 and the tube 124 have been inserted to the target position, the connection may be released by, for example, cutting the connection at the proximal ends 110, 126.

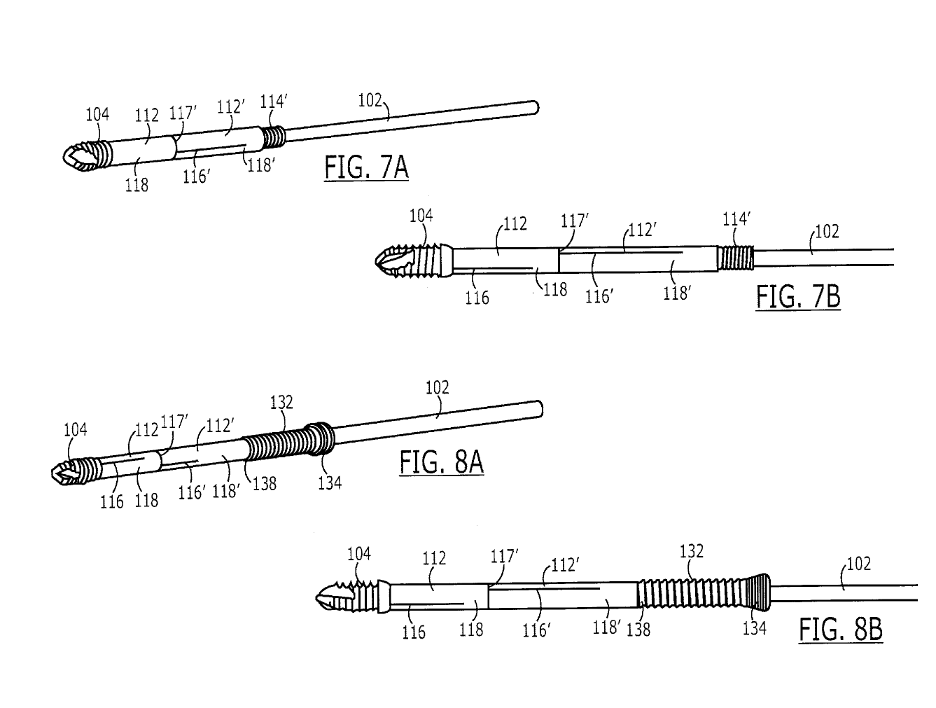

FIGS. 7-10 sequentially depict the process of the splaying of the arms 122 of the bushings 112 in accordance with a first exemplary method of the present invention. Initially, the rod 102 is screwed into the bone 10 by one of manual screwing and an automated screwing as is known to those skilled in the art. Once the threaded tip 104 has reached a target location in the bone 10, the first bushing 112 is advanced distally over the rod 102 until the distal end 117 comes into engagement with a flared proximal end of the threaded tip 104, as shown in FIGS. 7A and 7B. The second bushing 112′ is then advanced distally over the rod 102 until the threaded tip 114 is received within aims 122′ thereof. Embodiments of the present invention may employ bushings of any of a variety of configurations to maximize the bond strength between the bone fixation device 100 and the bone, as those skilled in the art will understand. For example, the number of bushings and/or the number of arms of the bushings employed in a bone may be varied in relation to the severity of the fracture and the level of bone decay. The number of bushings may be decided in advance of the performance of a bone fixation procedure by selecting a particular configuration of bushing(s) a plurality of choices to suit the particular injury to be treated. It is further noted that the lengths and number of bushings employed in a single procedure may be chosen so that, when inserted over the rod 102, the bushings will be housed within the bone 10 and will not protrude from the bone. In this manner, the cannulated screw 132 placed thereover is able to engage a bone plate 12 located adjacent the bone 10. Specifically, the threaded shaft 136 of the cannulated screw 132 engages a threaded bore 14 of the bone plate 12 to permit screwing into the bone 10, as those skilled in the art will understand. Furthermore, the size and number of bushings 112 used in a bone fixation procedure according to the present invention may be chosen so that, once the cannulated screw 132 has been screwed into a distal-most position in the bone 10 at which engagement of the head 134 and the bone plate 12 prevents further entry, the arms 122 and 122′ are splayed radially outward into the subchondral layer 18.

According to an alternate embodiment of the present invention, a locking mechanism may be provided to lock the cannulated screw over the bone plate, thus preventing movement of the rod 102 relative to the bone plate 12. As shown in FIG. 17, a cannulated screw 532 comprising a lumen 540 extending therethrough is formed substantially similar to the cannulated screw 132 with the exception of a locking mechanism provided on a head 534, as will be described in greater detail hereinafter. The head 534 is formed with a taper, with a circumference of a distal end 546 of the head 534 being smaller than a circumference of a proximal end 544. The head 534 further comprises a first threaded region 548 longitudinally separated from an engagement region 550. The threaded region 548 comprises threads 552 sized and shaped to engage threads of a bore in a bone plate (not shown) through which the cannulated screw 532 will be inserted. The engagement region 550, which lies proximally of and adjacent to the threaded region 540, permits engagement with a device used to screw the cannulated screw 532 into the bone plate (not shown). The engagement region 550 may be formed in the shape of a hexagon, although a plurality of other shapes facilitating screwing of the cannulated screw 532 may be employed.

A locking feature on the head 534 comprises a plurality of longitudinal slots 542 which extend substantially parallel to a longitudinal axis of a lumen 540, the slots 542 defining a series of arms 552. The slots 542 extend through the entire length of the head 532 and are open to the lumen 540. Furthermore, the slots 542 in this embodiment are substantially evenly distributed about a circumference of the head 532 so that an even pressure is applied to the rod 102 when inserted through the lumen 540, as will be described in greater detail below.

In use, the bone plate (not shown) is first situated over a target portion of the bone and secured thereto, as described earlier. The bone plate comprises a tapered bore (not shown), a circumference of which decreases from a proximal end to a distal end which lies adjacent to the bone. The tapered bore (not shown) further comprises an internal thread sized and configured to receive threads of the threaded region 548. In accordance with the method disclosed earlier, once the bushing 112 has been advanced over the rod 102 into the bone, the cannulated screw 532 is inserted over the rod. Threads of a shaft 536 of the cannulated screw 532 are then screwed past the tapered bore (not shown) so that insertion of the threaded region 548 into the tapered bore (not shown) radially compresses the head 534 as each arm 552 is pushed radially inward to apply pressure against the rod 102. Specifically, the reduction in diameter of the tapered bore (not shown) applies radially inward pressure to the head 534 as the head 534 is pushed distally therethrough deforming the arms 552 which deflect radially into the rod 102 locking the rod 102 in place against the screw 532.

In an alternate embodiment, the first bushing 112 and the second bushing 112′ may be deployed separately as deemed necessary by a user. In this embodiment, once the first bushing 112 has been moved to an expanded configuration in a bone 10, a second bushing 112′ may be advanced distally over the rod 102 until a distal end 117′ thereof comes into engagement with the first bushing 112, lying directly adjacent thereto. In an exemplary embodiment, dimensions of the second bushing 112′ may be substantially the same dimensions as the first bushing 112. However, in an alternate embodiment, the lengths of the arms 122′ of the second bushing 112′ may be of different lengths allowing the arms 122′ to splay outward at positions separated from one another longitudinally, as those skilled in the art will understand. In one embodiment, slots 116′ of the second bushing 112′ are formed out of alignment with the slots 116 of the first bushing 112. That is, the slots 116 of the first bushing are positioned radially about the rod 102 at a first set of angular orientations while the arms 122′ of the second bushing 112′ are rotated relative thereto to a second set of angular orientations different from the first set. Thus, as the arms 122′ splay outward, they are lodged in portions of the bone separate from those in which the arms 122 are lodged, increasing the strength of the bond between the device 100 and the bone 10.

Once the first and second bushings 112, 112′, respectively, have been advanced to their indicated positions, the cannulated screw 132 is advanced over the rod 102 to lie in engagement with a threaded portion 114′ at a proximal end of the second bushing 112′, as shown in FIGS. 8A and 8B. As indicated earlier, the lengths of the first and second bushings 112, 112′, respectively, are chosen so that, when the cannulated screw 132 is advanced over the rod, the head 134 remains external to the bone 10. In this manner, a different cannulated screw 132 may be employed to screw in the first bushing 112 and the second bushing 112′ to account for the different screw lengths required for the two bushings, as those skilled in the art will understand. As shown in FIG. 2, the head 134 preferably lies directly adjacent to an outer surface of the bone 10. A bone plate 12 is coupled to an outer surface of the bone 10 with the threaded bore 14 thereof in alignment with a point of entry for the rod 102. The bone plate 12 provides external stability to the rod 102 and aids in alignment of the rod 102 during the course of the bone fixation procedure. The threaded bore 14 formed in the bone plate 12 may, for example, be adapted to receive only a threaded shaft 136 of the cannulated screw 132 therethrough.

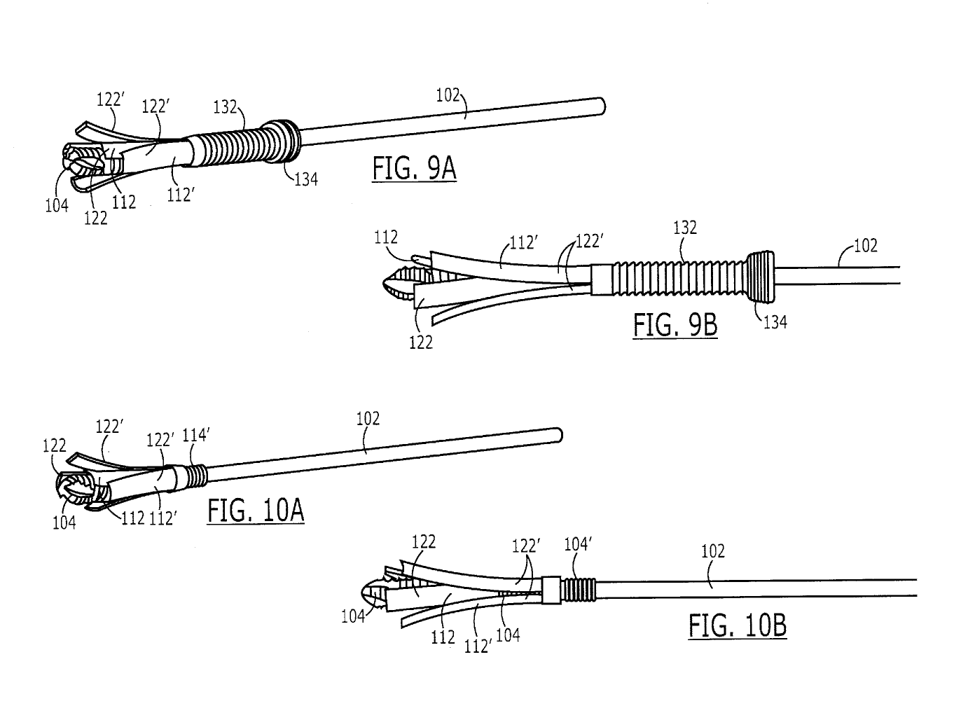

In an exemplary embodiment, the distal end 138 of the cannulated screw 132 comes into contact with the threaded portion 114′ located at a proximal end of the first bushing 112′ at a depth selected so that only a distalmost thread of the cannulated screw 134 is received in the bone 10. A remainder of the cannulated screw 132 remains external to the bone 10 prior to screwing thereinto. As shown in FIGS. 9A and 9B, screwing of the cannulated screw 132 in a first direction moves the first bushing 112 and second bushing 112′ distally along the rod 102. The distal pressure causes the first bushing 112 and second bushing 112′ to slide over the flared threaded tip 104 causing the arms 122 and 122′ to splay radially outward at an angle substantially equal to a taper angle thereof, thus increasing the surface contact area of each arm 122 and providing a rigid hold between the bone fixation device 100 and the bone. As the fracture in the bone 10 heals, bone tissue grows into any voids in the bone fixation device 100 and strengthens the bone 10 further, as those skilled in the art will understand. Subsequently, as shown in FIGS. 10A and 10B, the cannulated screw 132 may be removed from the bone 10 by screwing the screw 132 in the opposite direction out of engagement with the bone plate 12. The rod 102 is sized so that, when inserted to the desired depth within the bone, the proximal end of the rod 102 remains distal of a proximal surface of the plate 12 and does not project proximally therefrom.

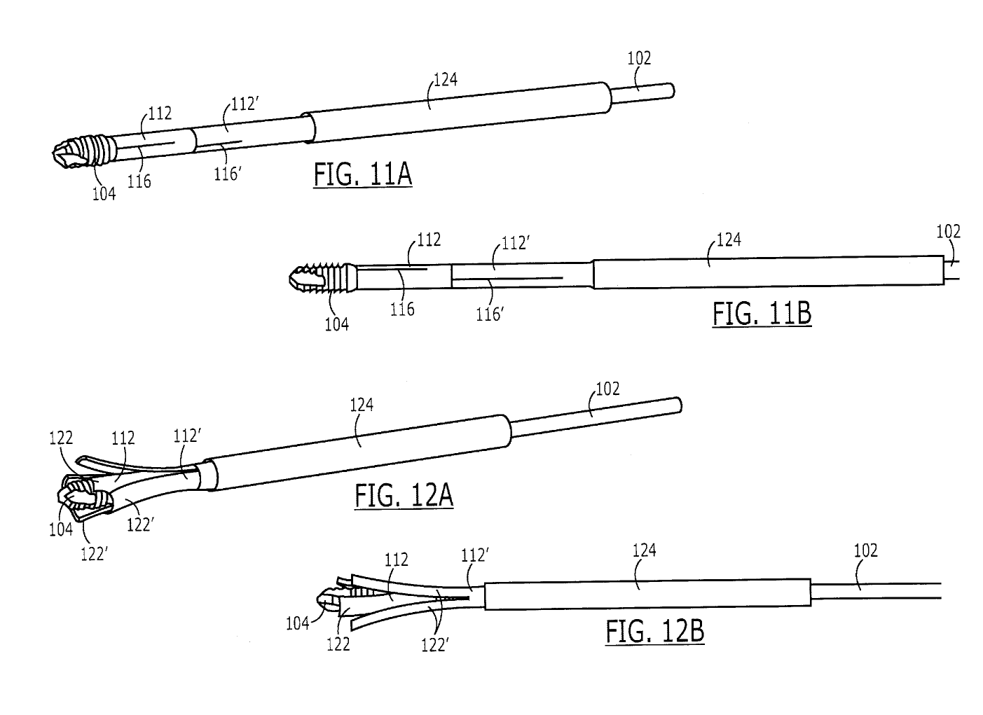

As shown in FIGS. 11 and 12, an alternate embodiment of the present invention bypasses the need to screw the cannulated screw 132 into the bone 10. In this embodiment, once the rod 102 and the bushings 112, 112′ have been advanced to target locations in the bone 10, the tube 124 is advanced distally over the rod 102 while a proximal portion thereof is remains external to the bone 10. A distally directed force is then applied to the rod 102 either manually or via a mechanism as known in the art causing the first and second bushings 112, 112′, respectively, to advance distally over the threaded tip 104 of the rod 102. As described above, this causes the arms 122, 122′ to splay outward. Once the arms 122, 122′ of the bushings 112, 112′, respectively, have been fully extended, the tube 124 may be removed from the rod 102. Forming the bushings 112, 112′ of a material visible under x-Ray allows the user to verify that the bushings have fully extended. Alternatively, the overall length of the device including the bushing and tube together with the rod may be compared to the distance by which the bushing and tube have moved over the rod to determine whether the bushing has been fully extended. As would be understood by those skilled in the art, the bushings need not be deployed in every case—this is an option. An alternative would be to introduce the rod 102, the bushings 112 and the tube 124 or the screw 132 into the bone without deploying the bushing. During the healing the fracture gap will increase because the Osteoclasts will remove damaged bone in the fracture zone before Osteoblasts will build it up again. Having a bigger fracture gap, leads to higher forces imposed on the rod 102. When a certain force is exerted on the rod 102, the rod 102 will slide proximally automatically deploying the bushings 112, 112′. The deployed bushings 112, 112′ will then increase the area of contact to withstand these higher forces. The sliding comes to a halt when the head comes into contact again with the shaft. This is generally after several mm.

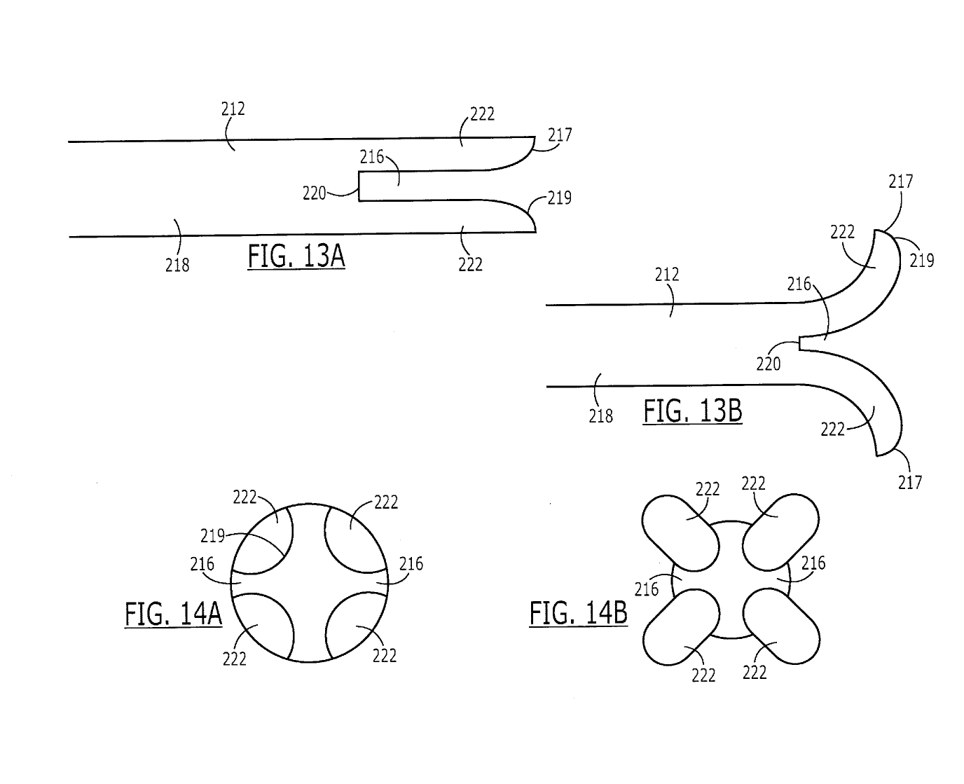

It is noted that various modifications may be made to the present application without deviating from the spirit and scope of the invention. In one example, as shown in FIGS. 13A-14B, a bushing 212 may be employed in place of the first bushing 112, comprising substantially the same elements. The bushing 212 extends from a proximal end (not shown) to a distal end 217 comprising a plurality of arms 222 defined by slots 216. The slots 216 extend from the distal end 217 of the bushing 212 to respectively termination points 220 located along a predetermined length of the body 218. The arms 222 may comprise substantially the same dimensions as the arms 122 with the exception of rounded ends 219 provided at the distal end 217 of each of the arms 222. The rounded ends 219 are formed with a rounded face along an inner diameter of the arms 222. Those skilled in the art will understand that the rounded ends 219 facilitate the sliding of the arms 222 distally over the threaded tip 104 of the rod 102 by providing a smooth interface without pointed edges therebetween. FIGS. 13B and 14B depict the arms 222 in the extended configuration splayed radially outward from the tip 104.

It will be apparent to those skilled in the art that various modifications and variations may be made in the structure and the methodology of the present invention, without departing from the spirit or scope of the invention. Thus, it is intended that the present invention cover modifications and variations of the invention provided that they come within the scope of the appended claims and their equivalents.

What is claimed is:

1. A device for treating a bone within a living body, comprising:
a rod extending from a proximal end which, in an operative configuration, remains external to the body to an increased diameter distal tip sized and shaped for insertion into a subchondral layer of the bone;
a first bushing slidably received over the rod, a distal portion of the first bushing comprising a plurality of longitudinally extending arms, the arms being separable from one another to spread radially outward from the rod as the first bushing is moved distally over the distal tip of the rod; and
a cannulated element slidably received over the rod, the cannulated element sized and shaped to engage a proximal end of the first bushing with a portion thereof located external to the bone and accessible to a user in the operative configuration, so that a distally directed force applied thereto moves the first bushing distally over the rod spreading the arms radially outward relative to the rod.

2. The bone fixation device of claim 1, wherein the arms of the first bushing are separated from one another by a plurality of slots extending proximally through a portion of the first bushing from a distal end thereof.

3. The bone fixation device of claim 1, wherein the increased diameter distal tip is threaded to permit screwing into the bone.

4. The bone fixation device of claim 1, wherein the first bushing includes a threaded proximal end.

5. The bone fixation device of claim 4, wherein the cannulated element is a cannulated screw, a proximal portion of the cannulated screw comprising a head with an outer diameter greater than an outer diameter of a shaft of the screw.

6. The bone fixation device of claim 5, wherein the head is selectively engageable with a device for screwing the cannulated screw into the bone.

7. The bone fixation device of claim 4, wherein the cannulated element is an elongated hollow tube comprising a lumen extending therethrough from a proximal end to a distal end, a diameter of the lumen being greater than an outer diameter of the rod.

8. The bone fixation device of claim 7, wherein a portion of the head is threaded, the head further comprising a plurality of longitudinally extending arms separable from one another to extend radially into the lumen.

9. The bone fixation device of claim 8, wherein the arms of the head are separated from one another by a plurality of slots extending through the head.

10. The bone fixation device of claim 1, wherein the increased diameter distal tip comprises a notch extending substantially parallel to a longitudinal axis of the rod.

11. The bone fixation device of claim 1, further comprising a second bushing slidably received over the rod, a distal portion of the second bushing comprising a plurality of longitudinally extending arms, the arms being separable from one another to spread radially outward from the rod as the second bushing is moved distally over the tip of the rod.

12. The bone fixation device of claim 11, wherein the arms of the second bushing are out of alignment radially about the rod with respect to the arms of the first bushing.

13. The bone fixation device of claim 11, wherein distal ends of the arms of the first bushing are rounded.

14. A method of treating a bone, comprising:
inserting a rod including a flared distal tip to a first target depth in a subchondral layer of a fractured bone;
positioning a first hushing over the rod, a distal portion of the first bushing comprising a plurality of longitudinally extending arms; and
advancing a cannulated element distally over the rod to engage a proximal end of the first bushing and move the first bushing distally over the flared distal tip of the rod so that the arms separate from one another and spread radially outward from the rod.

15. The method of claim 15, further comprising:
positioning a bone plate over the fractured bone, the bone plate comprising a tapered bore extending therethrough;
advancing the cannulated element distally through the tapered bore to engage the proximal end of the first bushing, wherein an increased diameter head at a proximal end of the cannulated element comprises a plurality of arms longitudinally extending arms, so that the arms separate from one another and extend radially inward to frictionally engage the rod.

16. The method of claim 15, wherein the rod is inserted into the bone until a wind-up of the rod increases to a tactile feedback level indicative of a positioning of the flared distal tip within the subchondral layer.

17. The method of claim 15, wherein a proximal end of the cannulated element is connected to a proximal end of the rod when the rod is inserted into the bone, further comprising the step of severing the connection between the cannulated element and the rod after insertion thereof to the target depth.
